(19) United States
(12) Patent Application Publication
(10) Pub. No.: US 2025/0241790 A1
(43) Pub. Date: Jul. 31, 2025

(54) EYE SYRINGE AND METHOD

(71) Applicant: Health Corporation of Tzafon Medical Center (R.A), PORIYYA ILLIT (IL)

(72) Inventor: Aseel NASSAR, Arraba (IL)

(21) Appl. No.: 18/870,682

(22) PCT Filed: May 31, 2023
(86) PCT No.: PCT/IL2023/050560

§ 371 Date: Dec. 1, 2024

(30) Foreign Application Priority Data
Jun. 2, 2022 (IL) 293586

(51) Int. Cl. A61F 9/00; A61M 5/32

(52) U.S. Cl. CPC A61F 9/0017; A61M 5/3245; A61M 5/3286; A61M 2205/3306; A61M 2210/0612

(57) ABSTRACT

An eye syringe includes an elongated handle. The handle includes a needle that, upon actuation from a retracted position, extends through an opening at a distal end of the handle so as to penetrate an eye of a subject at a predetermined angle. The distal end forms a concave tip shaped to match the contour of, and hold, an outer surface of the eyeball. The distal end has a protruding upper edge configured to be inserted behind an upper eyelid so as to distance the upper eyelid from the needle and hold the upper eyelid in place during the injection. The distal end may also have a lower edge configured to demarcate a border of a portion of the eye—such as the limbus—so as to accurately position the needle. A flap may move to cover the point of injection after the injection.

### FIELD OF THE INVENTION

The invention is in the field of eye surgery, and in particular, a syringe for intravitreal injection.

### BACKGROUND OF THE INVENTION

As a result of certain eye diseases and conditions, such as macular degeneration, diabetic retinopathy, retinal vein occlusion, uveitis, patients require intravitreal injections. Intravitreal injections often need to be repeated in chronic conditions, e.g., many patients require monthly eye injections whose purpose is to absorb fluids found in the retina. Most of the patients are above the age of 60 and have difficulty with mobility, for example because they are wheelchair bound, or they have chronic back pain which makes it difficult for them to lie on their back or in a reclining chair to receive the injection. Today the procedure for getting the injection is a long procedure taking on average ten minutes to fifteen minutes, even though the actual injection procedure does not exceed two minutes.

The following steps are presently taken for the whole procedure:
1. Entering the nurse's station to be identified and receive eyedrops as anesthesia.
2. Entering the physician's office to receive the injection.
3. Transferring the patient to the bed, typically from a wheelchair, such that this requires two assistants. Typically, the patient also has back, neck, and spinal pain so that they find it hard to lie down, which is the best position of the head for the injection.
4. After lying down, the bed has to be adjusted so that it is comfortable for the physician who gives the injection, for example raising or moving the bed to the side.
5. The physician opens the set of sterile gloves and determines (by communication with the patient and the computer system) which drug to be injected in which eye and whether complications arose during the previous injection.
6. The team (physician and nurse) opens the sterile set that includes pads, swabs, a speculum, a cup into which 10% iodine solution is poured, a syringe for injecting 4% iodine solution.
7. The nurse pours the 10% iodine solution into the cup and places it on the sterile set that had been opened.
8. The physician places a needle on a 20-gauge syringe and draws 4% iodine solution into it.
9. The nurse opens a syringe and hands it to the physician who places a 30-gauge needle on it and prepares the syringe for a small quantity (e.g., 0.05 ml) of the drug to be injected.
10. Sterilize the area surrounding the target eye occurs using a 10% iodine solution and then cover these surrounding areas of the eye with a sterile cover.
11. Place a sticker (e.g., Tegaderm™ sticker) to raise the eyelashes.
12. Place the speculum in the eye in order to the open the upper and lower eyelids and prevent blinking and to have a work area.
13. The physician puts drops from the iodine solution containing syringe containing 4% iodine solution into the eye.
14. Using the top of the top of the drug solution containing syringe, mark off 4 mm from the limbus in order to indicate the location of the injection.
15. Inject the drug solution and remove the needle.
16. Place a swab on the area of the injection for a few seconds.
17. Put antibacterial eyedrops and anesthetics into the eye.
18. Remove the speculum, sticker and sterile cover.
19. The patient sits up for a few minutes and returns to the wheelchair or leaves the office.

The above procedure takes a relatively long amount of time. In addition, sometimes significant physical effort is needed to move the patient. The patient is also not always willing or able to lie in the position that is optimal for the most precise injection. Furthermore, this procedure requires extensive preparations and use of instruments and supplies (speculum, syringes, stickers, sterile cover, 10% iodine solution). Moreover, sometimes the patient moves the eye during the injection, which is liable to cause damage, such as bleeding, perforation of the cornea or injury to the lens or a hole in the retina from the injection being too far to the posterior. Further, while a sticker is used to raise the eyelashes, they are generally more difficult to disinfect and contact between the eyelashes and the syringe needle may transfer bacteria onto and/or into the eye, resulting in infection.

Therefore, there is a need for an efficient, sterile intravitreal syringe to streamline the injection procedure and increase the comfort of the patient and the physician.

### SUMMARY OF THE EMBODIMENTS

The invention is in the field of eye surgery, and in particular, to a syringe for intravitreal injection.

Some embodiments relate to an eye syringe, comprising:
an elongated handle comprising a needle that, upon actuation from a retracted position, extends through an opening at a distal end of the handle so as to penetrate an eye of a subject at a predetermined angle, wherein the distal end of the handle comprises a distal section,
the distal end of the distal section forming a concave tip shaped to match and hold an outer surface of an eyeball of the eye,
the distal end of the distal section having a protruding upper edge configured to be inserted behind an upper eyelid so as to distance the upper eyelid from the needle and hold the upper eyelid in place during an injection;
the distal end of the distal section also having a lower edge configured to demarcate a border of a portion of the eye so as to accurately position the needle.

According to some embodiments, the distal section may be rotatable about a longitudinal axis of the handle. Additionally, and/or alternatively, the distal section and/or the handle may include a micro-camera configured to capture the border of the portion of the eye. The distal section and/or the handle may include a processor configured to process a visual signal captured by the micro-camera. The distal section and/or the handle may include a processor configured to provide an audio signal to indicate a border of a portion of the eye.

According to some embodiments, a lower edge of the distal end of the distal section may be configured to demarcate a limbus of the eye, and position the needle at an optimal location for injection e.g., at about 3-5 mm from the limbus. The lower edge of the distal end of the distal section may be configured to demarcate an upper portion of the limbus of the eye.

According to some embodiments, at least one component of the handle and/or distal section may be transparent and is configured to provide a user with a view of the needle, the eyeball or both. Each possibility is a separate embodiment.

According to some embodiments, the concave tip may be configured to immobilize the eye during the injection and/or retraction of the needle and/or to stabilize the syringe on the eyeball. The concave tip may include a rubber or silicone ring to facilitate immobilization of the eye and/or to stabilize the syringe.

According to some embodiments, the handle may house the needle or a portion thereof. According to some embodiments, the handle may include a needle actuator for actuating the needle. Optionally, the needle actuator may be protected by a cap.

According to some embodiments, the handle may include a port configured to receive a fluid to be injected into the eye. According to some embodiments, the handle may include a fluid reservoir. According to some embodiments, the fluid reservoir may be configured to be filled with the fluid through the port.

According to some embodiments, the fluid reservoir may be pre-filled with the fluid. According to some embodiments, the handle may be configured to receive a vial pre-filled with a fluid to be injected into the eye. Optionally, the fluid may be a gas and/or liquid. Optionally, the fluid is or includes a therapeutic agent.

According to some embodiments, the device may include a movable flap configured to cover a point of injection in a sclera of the eye through which the needle penetrated. The movable flap may be in a first position situated on a side portion of the distal end of the distal section before the injection, and may be configured to move to a second position distal to the distal end of the distal section after the injection and retraction of the needle. Optionally, a manual actuator may be configured to move the movable flap from a first position to a second position. Optionally, the movable flap may be configured to automatically move to a second position covering a point of injection upon retraction of the needle from an extended position to a retracted position.

Some embodiments relate to a method of performing an intravitreal injection of medication in an eye, the method comprising:
positioning an eye syringe against the eye so that an upper edge protruding out of a distal end of a rotatable distal section associate with a handle of the eye syringe is inserted under an upper eyelid of the eye, so that the distal end is configured to match a contour of the eye and thereby immobilize an eyeball of the eye, and so that a lower edge of a distal end of the distal section demarcates a limbus of the eye;
actuating an injection of a needle in a handle of the syringe and situated at a predetermined angle for injection through a sclera at a predefined distance from the limbus;
after injecting through the sclera and retracting of the needle back into the handle.

According to some embodiments, the demarcating may include a micro-camera configured to capture the limbus of the eye, a processor may be configured for processing a visual signal captured by the micro-camera, wherein the processor may be configured for providing an audio signal indicating the limbus of the eye. According to some embodiments, the demarcating may include positioning the needle at about 4 mm from the limbus.

According to some embodiments, the immobilizing may include contacting the eyeball with a rubber or silicone ring attached to the distal end of the distal section.

According to some embodiments, a movable flap of the handle may be moved to a position covering a point of injection to prevent escape of medication through the injection. According to some embodiments, a movable flap may be moved to the position from a previous position at a side of a distal end of the distal section.

According to some embodiments, the positioning of the syringe and actuating the injection may be performed using one hand.

According to some embodiments, the injecting may include dispensing a fluid from a pre-filled fluid reservoir. According to some embodiments, the injecting may include dispensing a fluid from a fluid reservoir configured to be filled with the fluid through a port on the handle. According to some embodiments, the injecting may include dispensing a fluid from a pre-filled vial.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. Various embodiments are herein described, by way of example only, with reference to the accompanying drawings, wherein:

FIG. 1 is a side view of an eye syringe, in accordance with some embodiments;

FIG. 2 is a top view of an eye syringe of FIG. 1, in accordance with some embodiments;

FIG. 3 is a sectional view of an eye syringe taken along line B-B of FIG. 2, in accordance with some embodiments;

FIG. 4 is an isometric view of an eye syringe of FIG. 1 taken from the side and rear, in accordance with some embodiments;

FIG. 5 is a side view of the eye syringe of FIG. 1 with a concave tip representing the eye of the patient, in accordance with some embodiments;

FIG. 6 is a top view of an eye syringe of FIG. 2 with a concave tip representing the eye of the patient, in accordance with some embodiments;

FIG. 7 is a sectional view of an eye syringe taken along line B-B of FIG. 6 with a concave tip representing the eye of the patient, in accordance with some embodiments;

FIG. 8 is an isometric view of an eye syringe from the side and rear, adjacent a schematic image of the front of an eye and an eyelid, in accordance with some embodiments;

FIG. 9 is a sectional view of the eye syringe identical to FIG. 2 with the flap in a first position, in accordance with some embodiments;

FIG. 10 is a sectional view of the eye syringe of FIG. 9 taken along line D-D, in accordance with some embodiments;

FIG. 11 is an enlarged view of a distal end of the eye syringe of FIG. 10, in accordance with some embodiments;

FIG. 12 is a sectional view of the eye syringe identical to FIG. 2 with the flap in a second position after injection and retraction of the needle, in accordance with some embodiments;

FIG. 13 is a sectional view of the eye syringe of FIG. 12 taken along line E-E with the flap in a second position after injection and retraction of the needle, in accordance with some embodiments;

FIG. 14 is an enlarged view of a distal end of the eye syringe of FIG. 13, in accordance with some embodiments;

FIG. 15 is a schematic illustration of an eye showing a 4 mm distance between a point of injection and a limbus, in accordance with some embodiments;

FIG. 16 is an illustration of an eye syringe with a cap, in accordance with some embodiments;

FIG. 17 is an illustration of an eye syringe, in accordance with some embodiments;

FIG. 18 is an illustration of an eye syringe, in accordance with some embodiments;

FIG. 19 is an illustration of an eye syringe, in accordance with some embodiments;

FIG. 20 is an illustration of an eye syringe, in accordance with some embodiments;

FIG. 21 is an illustration of an eye syringe, in accordance with some embodiments;

FIG. 22 is an illustration of an eye syringe, in accordance with some embodiments; and

FIG. 23 is a flow chart of a method, in accordance with some embodiments.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The following detailed description is of the best currently contemplated modes of carrying out the invention. The description is not to be taken in a limiting sense, but is made merely for the purpose of illustrating the general principles of the invention, since the scope of the invention is best defined by the appended claims.

The invention generally provides an eye syringe for use in intravitreal and other injections. According to some embodiments, the device may have a distal end that marks off a place of injection. According to some embodiments, the device may have a distal end which orients an angle of injection by the needle, for example a needle housed in the handle of the device. According to some embodiments, an upper part of the distal end may be configured to distance the upper eyelid from the needle. According to some embodiments, an upper part of the distal end may be configured to hold the upper eyelid in place during the injection and/or subsequent retraction of the needle. According to some embodiments, the distal end may form a concave tip configured to match the contour of the eyeball.

Advantageously, in certain embodiments the eye syringe may be configured to fixate the eye, demarcate a place of injection, properly orient the angle of the needle for an injection, and distance and hold the upper eyelid, there is no need for the patient to be placed onto a bed in a lying position, e.g., the intravitreal injection can be performed on a sitting subject. According to some embodiments, the device and method described herein may be easier on the patient by reducing the required physical movement, such as on and off a bed. Furthermore, advantageously, there is no need for a sterile cover to be used because contact by the needle with the surroundings of the eye is prevented. Additionally, advantageously, the eyelid is distanced from the area of the injection thereby obviating the need for a speculum. In addition, advantageously, the eyelashes are distanced from the needle, reducing the possibility of contamination of the needle, and thereby further obviating the need for stickers or an iodine solution.

Using the device and method disclosed herein, the needle is situated in the device itself in certain embodiments. Advantageously, this reduces the risk of infection for at least two reasons. First, contact is prevented between the needle and the eyelashes e.g., in case some of the eyelashes (which are not sterile) are not properly covered, as with prior art devices and methods. Thereby reducing the possibility of contamination of the needle by microorganisms and/or dirt from the eyelashes. Second, the risk of infection is further reduced since contact is prevented between the needle and the physician's gloves and/or other surfaces, and there is always the possibility that the physician unknowingly touched something not sterile. Certain embodiments of the device provide protection from any iatrogenic contamination (e.g., from microorganisms, such as bacteria, viruses, fungi, etc. and/or dirt, etc.) before the injection is performed. The disclosed device is for one time use. The device and/or components thereof may be disposable. This may further limit any risk of infection since one cannot be certain that sterilization is always 100% effective, and any iatrogenic contamination may infect a patient by mistake, resulting in an infection and even possibly loss of the eye.

According to some embodiments, the eye syringe may be structurally configured to demarcate a point about 4 mm from the limbus. Optionally, it may no longer be necessary for the physician to employ the head of a syringe to engage in a process of marking such a point off during the procedure. In addition, the eye syringe may be configured to fixate and/or immobilize the eye during injection. Optionally, this may reduce and/or prevent incidents in which the patient's eye moves in a manner that may cause damage to the eye during injection.

In accordance with certain embodiments, moreover, the point of injection, angle of injection, and/or the depth of penetration of the needle may be controlled precisely. Optionally, this may minimize complications, such as bleeding, perforations of the cornea and/or injury to the lens of the eye or to the retina posterior to the 4 mm point of injection.

For these reasons, and others outlined below, the device and method disclosed herein, in some embodiments, is safe, decreases complications and is faster to use than prior art devices and methods.

In accordance with certain embodiments, the eye syringe may include a handle, for example an elongated handle. According to some embodiments, the handle may include a distal section. Optionally, one or more components of the handle and/or the distal section may be transparent. The distal section may be transparent. The transparent distal section may be configured to provide a physician with a clear view of the needle, the eyeball or both.

According to some embodiments, the distal section may be rotatable about a longitudinal axis of the handle. Optionally, rotation of the distal section about a longitudinal axis of the handle may change the position of one or more components of the device relative to the upper eyelid, e.g., port, actuator, grip, etc. Optionally, rotation of the distal section about a longitudinal axis of the handle may facilitate distancing, holding and/or raising the upper eyelid while facilitating the use of the eye syringe. Optionally, rotation of the distal section about a longitudinal axis of the handle may facilitate avoidance of sensitive and/or unusual eye topography, e.g., clusters of blood vessels, previous injection sites, ulcers, cataracts, glaucoma, etc. Optionally, rotation of the distal section about a longitudinal axis of the handle may facilitate the convenience of the physician e.g., left-or right-handed, left or right eye, space limitations (such, as equipment, staff members, etc.), additional caregivers, etc. Optionally, rotation of the distal section about a longitudinal axis of the handle may facilitate comfort of the patient, e.g., avoidance of sensitive and/or unusual eye topography, obviating the need for the patient to move, lie down or hold their heads in an uncomfortable position, etc.

According to some embodiments, the handle may house within it a needle and/or or a portion thereof. The needle may be reversibly extended from the handle through the distal section. The distance that the needle may be extended from the handle may be predetermined, e.g., set to an optimal depth of penetration of the eye. Optionally, the needle may be reversibly extended from the handle by an actuator. The needle actuator may actuate the needle manually and/or automatically. The needle actuator may be activated by a button on the handle. The needle actuator may be protected by a cap. Optionally, upon actuation from a retracted position, the needle may extend through an opening at a distal end of the distal section. The needle and/or the actuator may be configured to penetrate a subject's eye at a predetermined angle and location. The position of the needle within the handle may not rotate upon rotation of the distal section about a longitudinal axis of the handle.

According to some embodiments, the distal end of the distal section may be configured to be positioned against the eye of the subject. The distal end may orient the needle at the proper angle for injection, e.g., at about 30° to the surface of the eye, sclera or protruding upper edge 4. The angle for injection may be selected to ensure that the injection is at a safe distance from the lens, thereby preventing damage to the lens during injection. The distal end may orient the needle such that the position of injection is predetermined. The position of injection may be predetermined from the placement of an edge of the distal end of the distal section at the limbus of the eye.

According to some embodiments, the distal end of the distal section may form a concave tip. The concave tip may be shaped to match a contour of an outer surface of an eyeball. The concave tip may be shaped to hold an outer surface of an eyeball of the eye. The concave tip may be configured to stabilize the syringe on the eyeball. The concave tip may include a rubber or silicone ring on the distal surface. The ring may be configured to cushion the contact of the concave tip with the eye. The ring may be configured to facilitate holding an outer surface of an eyeball. The ring may be configured to facilitate immobilizing the eye during injection and retraction of the needle. The ring may be configured to stabilize the syringe on the eyeball.

According to some embodiments, the distal end of the distal section may have a protruding upper portion or upper edge. The protruding upper edge may be configured to be inserted behind and/or under an upper eyelid. The protruding upper edge may be configured to distance the upper eyelid from the needle. The protruding upper edge may be configured to hold the upper eyelid in place during an injection and/or subsequent retraction of the needle. The distancing and/or holding the upper eyelid away from the needle may provide protection from any iatrogenic contamination (e.g., from microorganisms, such as bacteria, viruses, fungi, etc. and/or dirt, etc.).

According to some embodiments, the distal end of the distal section may include a lower edge. The lower edge may be configured to demarcate a border of a portion of the eye, thereby to accurately position the needle.

In some embodiments, the border of the portion of the eye is a limbus. As used herein, according to some embodiments, the term “limbus” may refer to the transition zone between the opaque sclera and the clear cornea, separating the conjunctival epithelium and the corneal epithelium. The limbus is about 1 mm to 2 mm wide and is often measured by the normal, gradual loss of transparency as the cornea extends toward the periphery of the eye. As used herein, according to some embodiment, the term “upper limbus” may relate to the outer periphery of the limbus.

The lower edge may be configured to demarcate the limbus of the eye so as to accurately position the needle. The lower edge of the distal end of the distal section may be configured to demarcate a border of the eye, such as the limbus, preferably, an upper portion of the limbus, of the eye. Thereby, positioning the lower edge to orient the needle in a desired position for injection, preferably, between about 3 mm to about 4.5 mm from the limbus. For example, the lower edge may be configured to orient the needle about 4 mm from the limbus for injection.

According to some embodiments, the distal section and/or the handle may include a micro-camera. The micro-camera may be configured to capture the border of the portion of the eye. In some embodiments, the border of the portion of the eye is a limbus and wherein the micro-camera is configured to orient the needle about 4 mm from the limbus for injection of a fluid into the eye.

According to some embodiments, the distal section and/or the handle may include a processor. The processor may be configured to process a visual signal captured by the micro-camera. The processor may be configured to recognize a border of the portion of the eye in a visual signal. The processor may be configured to recognize the limbus in a visual signal. The processor may be configured to recognize an upper portion of the limbus in a visual signal. The processor may be configured to provide an indication to the physician of correct placement of the concave tip on the eye. Optionally, a processor may be configured to provide an audio signal to indicate a border of a portion of the eye. The processor may be configured to provide an indication to the physician of how close they are to the correct position of the eye syringe on the eyeball, e.g., an audio signal increasing in pitch and/or speed on approach to the upper portion of the limbus as recognized in a visual signal.

According to some embodiments, the handle of the eye syringe may include a port. The port may be configured to receive a fluid and/or vial pre-filled with a fluid. The port may be configured to receive a fluid to be injected into the eye. The fluid may be a gas or liquid. The fluid may be and/or may include one or more therapeutic agents.

According to some embodiments, the handle may include a fluid reservoir. The fluid reservoir may be fluidically connected to the needle. The fluid reservoir may be associated with the needle actuator. The fluid reservoir may be pre-filled with a fluid. The fluid reservoir may be pre-filled with a fluid immediately prior to use. Optionally, eye syringe may be provided with the fluid reservoir pre-filled with a fluid. The fluid reservoir may be configured to be filled with a fluid through a port on the handle. The handle may be configured to receive a vial pre-filled with a fluid to be injected into the eye.

According to some embodiments, handle and/or the distal section may include a movable flap. The moveable flap may be configured to cover a point of injection in a sclera of the eye through which the needle penetrated. The moveable flap may be configured to have two positions. The moveable flap may be in a first position before the injection and may be configured to move to a second position distal to the distal end of the distal section after the injection and retraction of the needle. Optionally, in the first position the movable flap may be situated on a side portion of the distal end of the distal section. Optionally, a manual actuator may be configured to move the movable flap from a first position to a second position. The movable flap may be configured to automatically move from to a second position covering a point of injection upon retraction of the needle from an extended position to a retracted position.

According to some embodiments, the eye syringe may include an activator. The activator may be required to be activated, e.g., by pushing, pulling, rotating, pressing, etc., prior to use. The activator may be a safety feature. The activator may prevent accidental discharge of the needle and/or fluid. The activator may maintain the sterility of the syringe and/or needle prior to use.

According to some embodiments, the eye syringe and/or components thereof may be composed of one or more materials. Optionally, some of the materials may be selected to provide a comfortable grip, professional look, protection for the components, etc.

The principles and operation of an eye syringe and method for intravitreal injections may be better understood with reference to the drawings and the accompanying description.

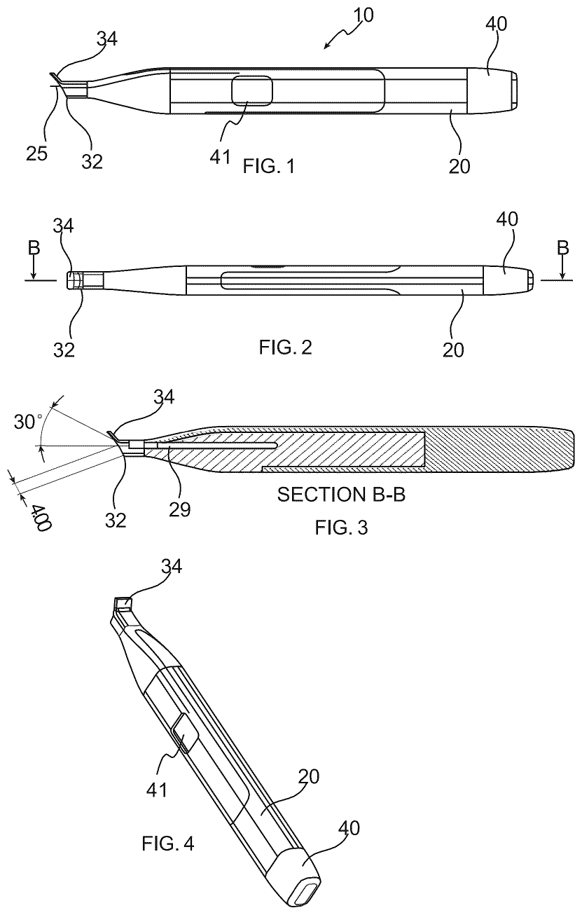

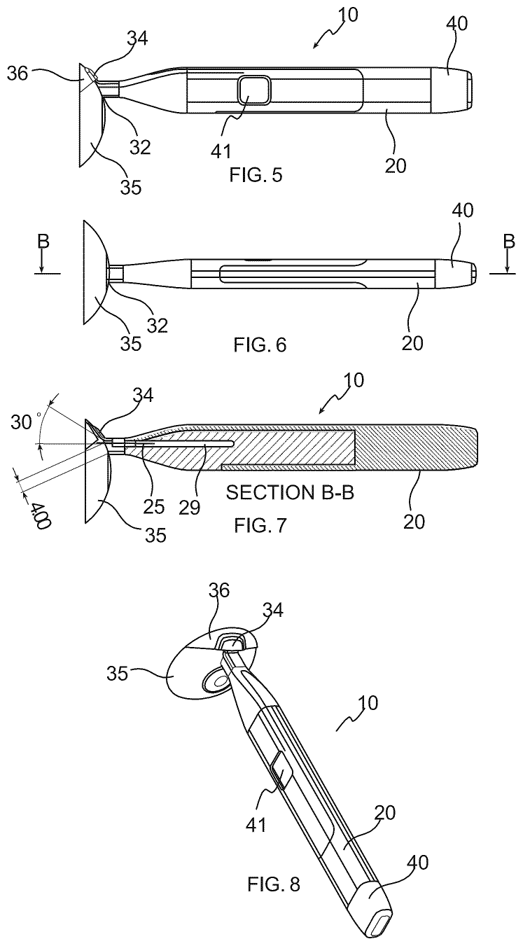

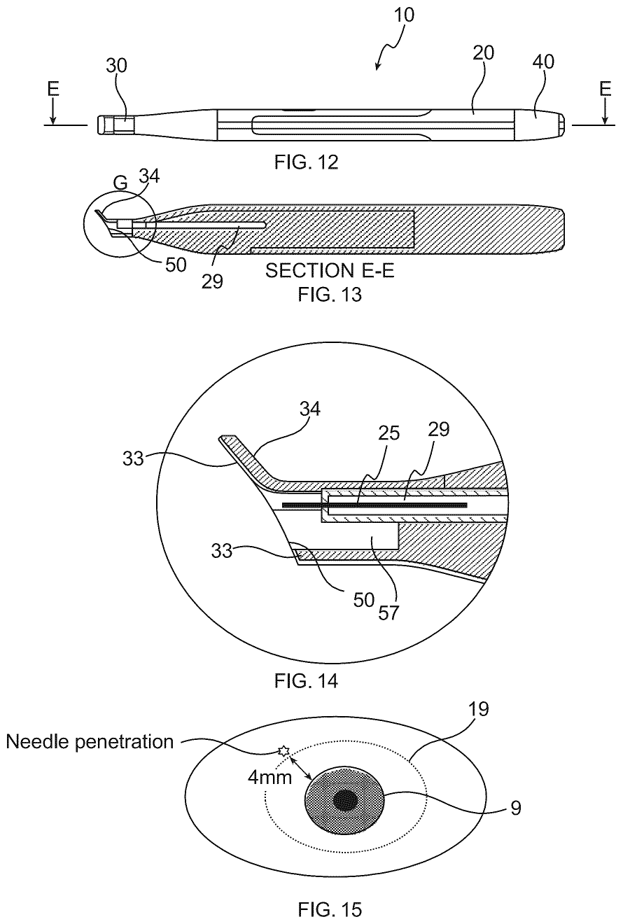

Reference is now made to FIGS. 1-16, which show an eye syringe 10 include a handle 20. The handle 20 has a proximal end 40 and a distal end 30. The syringe 10 comprises a handle 20, for example an elongated handle. Handle 20 includes a needle 25 or a portion thereof. In certain embodiments, the handle 20 houses the needle 25 or a portion thereof. In another embodiment, needle 25 is flush against an external wall of the handle 20. The needle 25 may be positioned within a housing of the handle 20 in a fixed manner at a specific location within the handle 20 and at a specific angle. The handle includes an actuator button 41. Actuator button 41 is configured to activate an actuator (not shown) associated with needle 25.

According to some embodiments, needle 25 may be oriented so as to be parallel with one or more longitudinal outer wall surfaces of the handle 20. According to some embodiments, the needle 25 may be positioned so as to be non-parallel to the outer wall surfaces of the handle 20. Optionally, the needle 25 is oriented at an angle such that the distal end 30 of the handle 20 is placed adjacent the eye in a particular manner as described herein. The needle 25 is configured to extend forward and to inject a fluid into the eye at the precise optimal position for injection in the eye, for example at the optimal position in the sclera of the eye about 4 mm from the limbus (which is the junction between the sclera and the cornea).

Reference is now made to FIGS. 1-4 and FIGS. 9-14, which show that the distal end 30 of handle 20 may form a concave tip 33 that is shaped to match and hold an outer surface of an eyeball of the eye.

Reference is now made to FIGS. 1-4, FIG. 8, FIG. 10, FIG. 11, FIG. 13 and FIG. 14, which show that the distal end 30 may also have a protruding upper edge 34. The protruding upper edge is configured to be inserted behind an upper eyelid so as to distance the upper eyelid from the needle and hold the upper eyelid in place during an injection. As seen from FIG. 10, FIG. 11, FIG. 13 and FIG. 14, the upper edge 34 may form an upper part of the concave tip 33.

The concave tip 33 of distal end 30 may have a concave shape to match the contour of the eyeball and is configured to be placed against the contour of the eyeball 35. The protruding upper edge 34 is configured to provide additional support to the concave tip 33 for holding the upper eyelid. In some embodiments, the protruding upper edge 34 is planar. In some embodiments, the protruding upper edge 34 protrudes upward between about 1 mm and about 5 mm, between about 5 mm and about 10 mm or between about 10 to about 12 mm. Each possibility is a separate embodiment.

Reference is now made to FIG. 3, which shows a protruding upper edge 34 extends so as to be at about 30° from the center of handle 20 where the needle 25 is positioned or from an axis extending from the needle 25.

The distal end 30 of the handle 20 may be shaped to match and hold an outer surface of an eyeball of the eye, in some cases all or substantially all or a majority of the outer surface of the eyeball or of the visible outer surface of the eyeball. The outer surface of the eyeball 35 as depicted in FIGS. 5-8.

Reference is now made to FIG. 8, which shows an upper portion 36 of the area denoting the eyeball 35 represents the upper eyelid covering the eyeball 35. Protruding upper edge 34 may be configured to be inserted behind an upper eyelid 36 so as to distance the upper eyelid 36 from the needle 25 and hold the upper eyelid 36 in place during the injection (typically including the actual injection by the needle 25 and the subsequent retraction of the needle 25). The distal end 30 and, in particular, its concave tip 33 may be configured to immobilize or fixate the eye during the injection and retraction of the needle 25.

Reference is now made to FIG. 5, FIG. 7 and FIG. 11, which show the distal end 30 of handle 20 may also have a lower edge 32 that—when the syringe 10 is in use—is lower than needle 25 within handle 20, for example a lower edge 32. The lower edge 32 may be configured to demarcate a border of a portion of the eye so as to accurately position needle 25 for injection. Accordingly, lower edge 32 may be about 4 mm below where needle 25 exits the lower end 30 of handle 20. In some embodiments, these 4 mm may be measured along a diagonal, as seen from FIG. 7.

Needle 25 or a portion thereof may be positioned within housing 20 so that it is a specific distance from a particular edge or portion of the distal end 30 of handle 20, for example from a lower edge 32, of distal end 30 of handle 20. For example, the needle 25 may be positioned so as to be a certain distance, for example a certain number of mm, for example mm about 3 mm, about 3.5 mm, about 4 mm or about 4.5 mm, from an edge of the concave tip 33, for example a lower edge 32, of the distal end 30 of handle 20.

Syringe 10 may be kept in a sterilized bag until use. Once opened, the syringe 10 is directly positioned on the eye within seconds. As a result, needle 25 is not exposed to the air in for a significant amount of time before being injected into the eye. This reduces the risk of infection. In the prior art, in contrast, the needle is exposed to the air (and potentially to contact with the physician's gloves or any unsterilized instrument or the eyelashes of the patient) for a relatively long time because it is necessary to open all the equipment and then start a five to six minute process (i.e., sterilize the face, cover the eyelashes, apply a medical solution, position the speculum, iodine solution, mark the injection area, etc.) before giving the injection, during which time the needle is exposed.

Likewise, in the prior art, the injected medication itself is also exposed to the air when the cap on the syringe is opened to connect a 30-gauge needle, whereas in syringe 10, the medication is embedded within in fluid reservoir within handle 20 since e.g., the medication is in a vial 29 within the handle and already connected to the needle 25.

Intravitreal injection procedures are difficult for elderly patients to endure, since many of them have back problems, back pain, or other health issues, and the procedure requires them, at least using prior art devices and methods, to be in an uncomfortable position for a relatively long time. However, with the eye syringe described herein, the patient's discomfort is reduced, and the duration of the procedure is shortened.

By virtue of the needle's position within handle 20 and because the lower edge 32 of the distal end 30 of handle 20 has demarcated the limbus before the injection, needle 25 is oriented precisely at the correct angle and in the correct position to be extended for the injection. Upon actuation of needle 25 from a retracted position (which may be by means of a needle actuator (not shown) for actuating the needle manually or which may be an automatic actuator (for example by a button accessible from the outside of the handle) triggered by one of a number of conditions, such as positioning the concave tip against the eyeball in a certain way with a certain force or after a certain number of seconds). Needle 25 is configured to extend through an opening at the distal end 30 of the handle 10 so as to penetrate a subject's eye at a predetermined optimal location of the sclera. Consequently, the distal end 30 is configured to demarcate the limbus so as to determine an optimal needle penetration location in the sclera of the eye e.g., about 4 mm from the limbus, which is the junction of the cornea and the sclera.

In some embodiments, since the perimeter of the iris is behind the limbus, and since if one looks at the eye one sees the iris through the cornea, the distal end 30 of handle 20, for example a lower edge 32 of distal end 30, is configured to demarcate a perimeter of the iris of the eye

The optimal location for the injection is in the sclera of the eye about 4 mm from the limbus. The optimal location is “about 4 mm” from the limbus to avoid contacting the lens and damage it e.g., at 3 mm from the limbus, which would require emergency surgery. Furthermore, if the injection were to be made at 5 mm distance from the limbus, the injection could create a hole in the retina, which could produce retinal hemorrhage or retinal detachment, also requiring an emergency surgery. In some embodiments, the syringe 10 is configured to position the needle 25 at an optimal location that is about 4 mm (i.e., the distance from the limbus is between about 3.6 mm to about 4.4 mm).

Another advantage of syringe 10 stems from the fact that not all patients have the same corneal diameter. Certain prior art intravitreal injection guides utilize one-size-fits-all instruments to perform the injection and utilize a measurement defined from a center of the cornea. Since corneal diameters differ, this method may result in injection in an incorrect position.

In contrast, in certain embodiments, syringe 10 is configured to position the needle 25 at the optimal position of penetration of the sclera by measuring about 4 mm from the demarcated limbus, which is unaffected by the size of the cornea. The disclosed syringe, in some embodiments, achieves this because syringe 10 is configured to allow a physician to demarcate the limbus by positioning the lower edge 32 of the distal end 30 of the handle at the limbus. This automatically positions the point of injection about 4 mm from the limbus without regard to the corneal diameter. Additionally, using syringe 10 for the demarcation of the limbus occurs simultaneously with immobilization of the eyeball and distancing the upper eyelid from the needle 25.

Since the limbus is a junction of the sclera and the cornea, and since the cornea is substantially annular or substantially elliptical, the limbus is substantially annular or substantially elliptical. The edge, for example the lower edge 32 of the distal end 30 of the handle 20 may be configured to demarcate the limbus of the eye, and in some embodiments to an upper portion of the limbus. As seen from FIG. 15, since the limbus is a substantially annular or annular or substantially elliptical or elliptical border, the optimal positions 19 for injection are about 4 mm from the limbus 9 and comprise a series of positions 19 that substantially form a circle or ellipse, as shown in FIG. 15. If one views the sclera as a round or elliptical clock, physicians typically choose a point that is about 2 o'clock for the left eye and 10:00 for the right eye along this series of optimal positions 19. However, this preference is mainly a convenience, and any other parts of the sclera that are about 4 mm from the limbus 9 along the series of optimal positions 19 may be used for intravitreal injection.

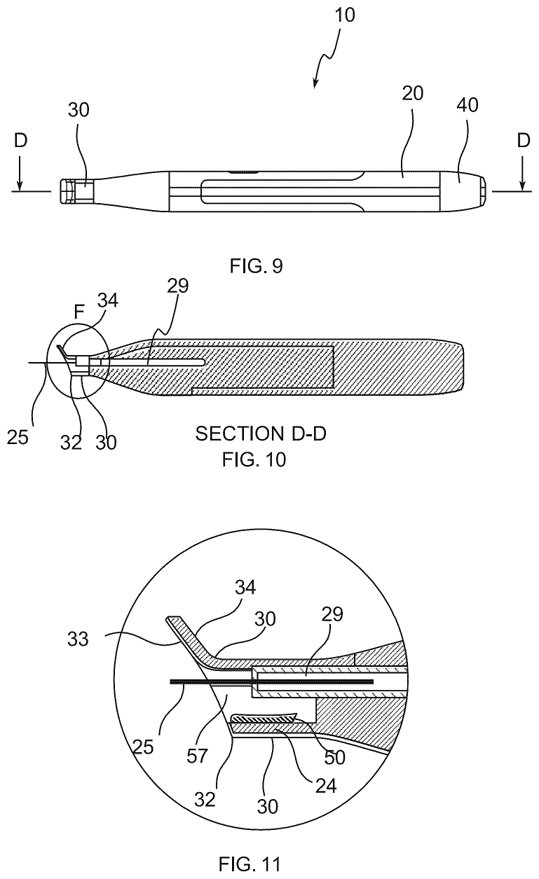

Reference is now made to FIGS. 9-14, which show eye syringe 10 may include a movable flap 50 that is configured to immediately cover the point of injection of needle 25 in the eye, for example in the sclera of the eye, after retraction of the needle 25 after the injection. The point of injection is the point on the sclera through which the needle 25 penetrated during the injection. In some embodiments, the movable flap is actuated automatically. In other embodiments, a manual actuator is configured to move the movable flap from a first position, best seen in FIG. 10 and FIG. 11, to a second position best seen in FIG. 13 and FIG. 14. The purpose of covering the point of injection is to prevent the injected medication from escaping back out of the eye (called drug reflux). This action could be undertaken by a manual act of the physician in applying a swab to the area of the injection for a few seconds, however, in certain embodiments, this action is obviated by the flap 50 of syringe 10.

In an automatically actuated embodiment, the movable flap 50 is configured to automatically move from a first position (best seen in FIG. 10 and FIG. 11) before the injection of the medication into the eye to a second position (best seen in FIG. 13 and FIG. 14) covering the point of injection after the injection and retraction of needle 25. In the second position, flap 50 covers the point of injection of needle 25 upon retraction of the needle 25 from an extended position to the retracted position, as shown in FIG. 13 and FIG. 14. As can be seen from FIG. 14, the second position of the flap 50 is distal to most of the distal end 30 (all except for the protruding edge 34) and may be located in a pocket 57 of the concave tip of distal end 30.

Reference is now made to FIG. 11, which best shows, in the first position of movable flap 50, the flap 50 is situated at a lower side 24 of distal end 30 or on a side 24 of a pocket 57 inside the distal end 30 of the handle 20. The mechanism for moving flap 50 from the first position to the second position shown in FIG. 14 which may, in one non-limiting example, comprise a spring (not shown) that moves flap 50 to its second position when an actuator moves a blocking element (not shown), which, in one non-limiting implementation, is a pin (not shown) located distal to flap 50. In the first position of flap 50, the blocking element blocks flap 50 from advancing from the first position to the second position. In the second position, the blocking element is deactivated or no longer in a position to perform blocking. The mechanism for moving flap 50 may be triggered manually and/or automatically.

In other embodiments, flap 50 may be external to the handle 20 in a sleeve or protected lumen adjacent to an outer wall of handle 20 when flap 50 is in a first position.

In some embodiments, distal end 30 of handle 20 of syringe 10 does not have any sharp edges that can accidentally scratch or otherwise damage the cornea of the patient's eye. The distal end may further be associated with a rubber or silicone layer or ring, which further protects the eye from damage. This is an advantage of certain prior art instruments that do have sharp edges.

In some embodiments, the eye syringe includes an elongated handle 20 housing a needle 25 or a portion thereof that, upon actuation from a retracted position, extends through an opening at a distal end 30 of the handle 20 so as to penetrate a sclera of a subject's eye at a predetermined angle, e.g., about 30° to the surface of the eye (or, in accordance with some embodiments, to protruding upper edge 34, which is configured to contact the surface of the sclera), to distance the needle from the lens, and thereby prevent damage to the lens during injection. The distal end 30 may have or form a concave tip 33 shaped to match a contour of an outer surface of the eyeball and thereby hold the outer surface of an eyeball of the eye fixed. The distal end 30 has a protruding upper edge 34 configured to be inserted behind an upper eyelid 36. The upper edge 34 may form an upper portion of the concave tip 33. The protruding upper edge 34 is considered “upper” when syringe 10 is in use and positioned against the eyeball of the patient. The protruding upper edge 34 is configured to distance the upper eyelid 36 and the eyelashes from the needle 25, thereby reducing or preventing contamination of the needle.

In some embodiments, syringe 10 includes a movable flap 50 configured to move to a second position (from an initial position for example an initial position at a lower side 24 of distal end 30 or a lower side of a pocket 57 at distal end 30) so as to cover a point of injection in the sclera through which the needle penetrated after the needle 25 is injected and retracted, as shown in FIG. 13 and FIG. 14. In some embodiments, the movable flap 50 may start in a first position at a side portion of the handle 20 and may be configured to move to a second position that is more distal after the retraction of the needle 25 so as to cover an opening at the distal end. Distal end 30 may have a lower edge 32 configured to demarcate a limbus of the eye so as to orient the needle 25 for penetration of a sclera of the eye at a position that is about 4 mm from the limbus.

In some embodiments, handle 20 may include a fluid reservoir 29, such as a vial for holding a medication prior to injection. The fluid reservoir 29 is proximal to the needle 25, as seen in FIG. 3 and FIG. 7.

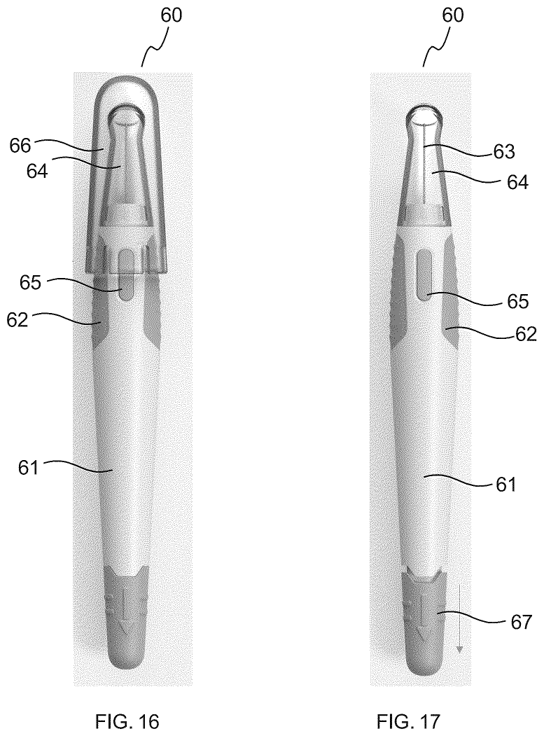

Reference is made to FIG. 16, which shows an illustration of an eye syringe with a cap, in accordance with some embodiments. Eye syringe 60 may include a variety of materials, such as a smooth handle 61 and one or more rubber grips 62. The distal section 64 of handle 61 is transparent. The handle includes an actuator button 65 to actuate the needle located proximal to the one or more rubber grips 62. Such an arrangement may facilitate one handed use of the eye syringe. The one or more rubber grips 62 may provide a comfortable area to rest the physician's fingers and/or may provide a good grip to reduce and/or prevent slippage during use. The palm of the physician's hand may be supported on the patient. The physician's second hand may assist in holding the patient's head still and/or in position. The eye syringe 60 includes a cap 66 which protects the sterility of the eye syringe and prevents accidental activation of the needle actuator 65.

Reference is made to FIG. 17, which shows an illustration of an eye syringe, in accordance with some embodiments. According to some embodiments, the eye syringe can be activated prior to use, by pushing, pulling, rotating, pressing, etc., an activator 67. Each possibility is a separate embodiment. The activator may be a safety feature to prevent accidental discharge of the needle and/or fluid. The activator may maintain the sterility of the syringe prior to use. Optionally, activating the activator 67 may lower the needle 63 from within the handle 61 to a stand-by position prior to use.

According to some embodiments, the distal section 64 and/or the needle 63 may be removable and/or disposable. The distal section 64 and/or the needle 63 may be replaceable prior to and/or after each use. The handle may be reusable.

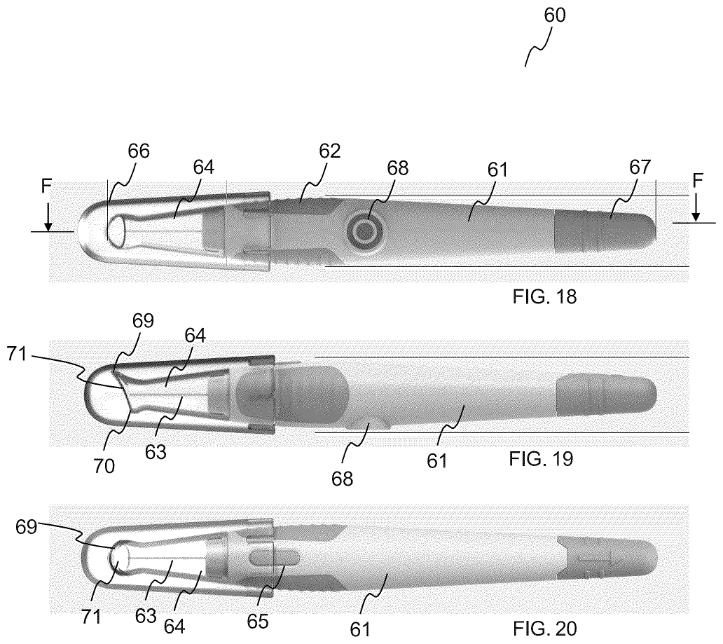

Reference is made to FIG. 18, FIG. 19, and FIG. 20, which show illustrations of an eye syringe, in accordance with some embodiments. According to some embodiments, handle 61 may include port 68 for filling a fluid reservoir (not shown). The fluid reservoir may be configured to be filled with a fluid through port 68 on handle 61. The fluid reservoir may be pre-filled with a fluid. The fluid reservoir may be pre-filled with a fluid immediately prior to use. Optionally, eye syringe 60 may be provided with the fluid reservoir pre-filled with a fluid. The fluid reservoir may be configured to accept a fluid filled vial. The fluid reservoir may be configured to accept a fluid filled vial at port 68. Optionally, fluid reservoir may be fluidically connected to needle 63.

According to some embodiments, distal section 64 may be rotatable about a longitudinal axis F-F of handle 61. Optionally, rotation of distal section 64 about a longitudinal axis of handle 61 may change the position of one or more components of the syringe relative to the upper eyelid, e.g., port 68, actuator 65, grip 62, etc. Optionally, rotation of distal section 64 about a longitudinal axis of handle 61 may facilitate distancing, holding and/or raising the upper eyelid while using of eye syringe 60. Optionally, rotation of the distal section 64 about a longitudinal axis of handle 61 may facilitate avoidance of sensitive and/or unusual eye topography, e.g., clusters of blood vessels, previous injection sites, ulcers, cataracts, glaucoma, etc. Optionally, rotation of distal section 64 about a longitudinal axis of handle 61 may facilitate the convenience of the physician e.g., left-or right-handed, left or right eye, space limitations (such, as equipment, etc.), additional caregivers, etc. Optionally, rotation of the distal section 64 about a longitudinal axis of handle 61 may facilitate comfort of the patient, e.g., avoidance of sensitive and/or unusual eye topography, obviating the need for the patient to move, lie down or hold their heads in an uncomfortable position, etc.

According to some embodiments, the distal end of the distal section 64 may form a concave tip 71. Optionally, distal end of the distal section 64 may have a protruding upper portion or upper edge 69. The protruding upper edge 69 may be configured to be inserted behind and/or under an upper eyelid. The protruding upper edge 69 may be configured to distance the upper eyelid and/or the eyelashes from the needle 63. The protruding upper edge 69 may be configured to hold the upper eyelid in place during an injection and/or subsequent retraction of the needle. The distancing and/or holding the upper eyelid away from the needle 63 may provide protection from any iatrogenic contamination (e.g., from microorganisms, such as bacteria, viruses, fungi, etc. and/or dirt, etc.).

According to some embodiments, the distal end of distal section 64 may include a lower edge 70. Optionally, lower edge 70 may be configured to demarcate a border of a portion of the eye so as to accurately position needle 63. Optionally, lower edge 70 may be configured to demarcate the limbus of the eye so as to accurately position needle 63. Optionally, lower edge 70 may be configured to demarcate an upper portion of the limbus of the eye. The lower edge 70 may be configured to orient needle 63 about 3 mm to about 4.5 mm from the limbus for injection of a fluid into the eye. Optionally, lower edge 70 may be configured to orient the needle 63 about 4 mm from the limbus for injection of a fluid into the eye.

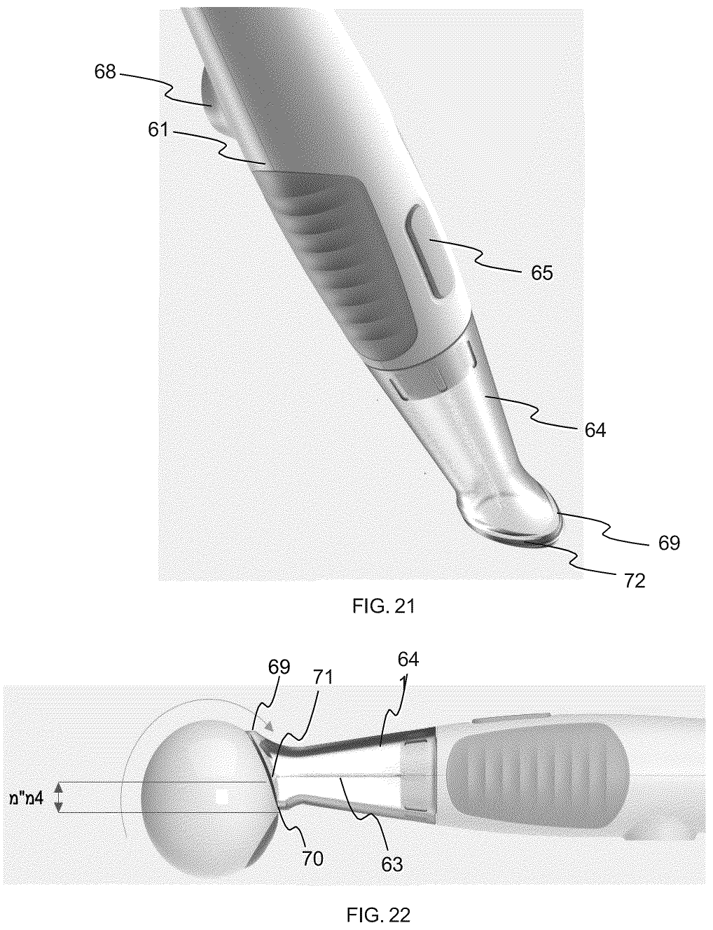

Reference is now made to FIG. 21 and FIG. 22, which show illustrations of an eye syringe, in accordance with some embodiments. According to some embodiments, the distal section 64 may be transparent to provide a physician with a clear view of the needle 63, the eyeball or both.

According to some embodiments, the distal end of the distal section 64 may form a concave tip 71, which may act as a speculum. The concave tip 71 may be shaped to match a contour of an outer surface of an eyeball. The concave tip 71 may be shaped to hold an outer surface of an eyeball of the eye. The concave tip 71 may include a rubber or silicone ring 72 on the distal surface. The ring 72 may cushion the contact of the concave tip with the eye. The ring may facilitate holding an outer surface of an eyeball.

According to some embodiments, the distal end of the distal section 64 may have a protruding upper portion or upper edge 69. The protruding upper edge 69 may be configured to be inserted behind and/or under an upper eyelid. The protruding upper edge 69 may be configured to distance the upper eyelid and/or the eyelashes from the needle 63. Optionally, lower edge 70 may be configured to orient needle 63 about 4 mm from the limbus for injection of a fluid into the eye.

Additionally, and/or alternatively, distal section 64 may be configured to enclose needle 63 prior to injection, during injection and after retraction of the needle from the eye. Optionally, concave tip 71 of distal section 64 may seal against an outer layer of the eyeball prior to injection, protecting the needle and preventing contamination from external sources.

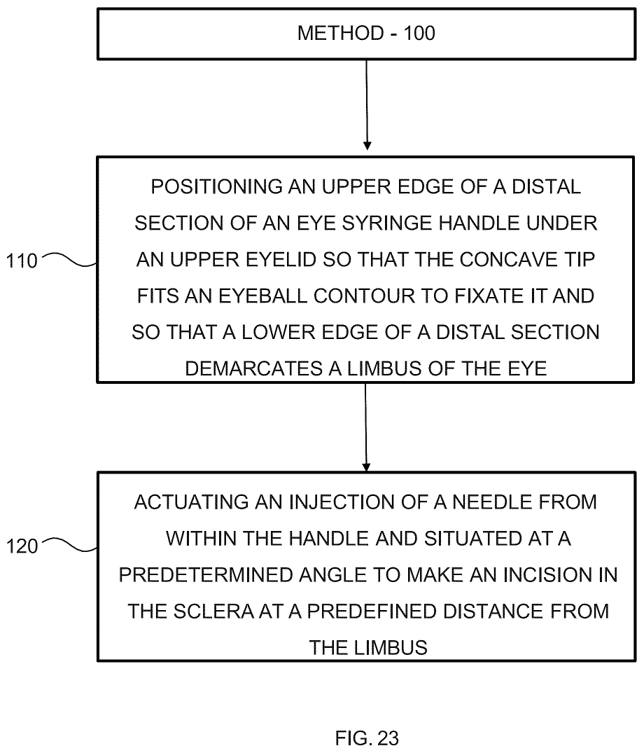

As seen in the flow chart of FIG. 23, another embodiment is a method 100 of performing an intravitreal injection of medication in an eye. In some embodiments, the method 100 may be performed while the patient is seated in a chair, or alternatively while in a wheelchair or alternatively while lying on a bed, and there is no requirement to have the patient lie down on a bed or assume a particular position.

Method 100 may include a step 110 of positioning an eye syringe against the eye so that an upper edge 34 protruding out of a distal end 30 of a handle of the eye syringe is inserted under an upper eyelid of the eye, so that the distal end (for example a concave tip 33 of the distal end 30) is configured to match a contour of and thereby immobilize an eyeball of the eye, and so that a lower edge 32, of a distal end 30 of the handle demarcates a limbus of the eye.

Another step 120 of method 100 may be actuating an injection of a needle of the handle, for example a needle that is housed in the handle of the syringe and situated at a predetermined angle for injection through the sclera (and into the vitreous at a predefined distance from the previously demarcated limbus). For example, the physician places the distal concave tip against the eyeball with the edge, for example the lower edge, of the distal portion at the limbus such that when the needle exits the handle is injected about 4 mm from the limbus.

Since syringe 10 does not require a speculum, method 100 may be performed without using a speculum to open the eyelids. Not having to utilize a speculum or equivalent instrument avoids the significant risk of ptosis of the upper eyelids from the injection, a risk which prior art devices and methods do incur. Furthermore, in some embodiments, normal use of syringe 10—and performance of the steps of method 100—is normally accomplished with one hand and method 100 may be performed using only one hand. This allows the physician to use the other hand to fixate the head of the patient and thereby relax the patient and/or limit head movement.

The method 100 may further comprise moving a movable flap 50 of the handle 20 to cover the point of the injection after the needed is retracted back into the handle 20. After the injection through the sclera, the movable flap 50 of the handle 20 may move to a position covering the point of injection after the retraction of the needle 25 back into the handle. The flap is automatically held against the eye (at the point of injection and its surrounding area) to prevent escape of medication through the injection.

Another step of method 100 may comprise moving the movable flap 50 to a second position covering the point of injection from a first position at a lower side of distal end 30 of handle 20 or at a lower end of a pocket 57 within the distal end 30 of the handle 20.

In some embodiments, step 110 and step 120 of method 100 may be performed using one hand.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

The terms “vial”, “cartridge”, “ampule” and “ampoule” are used interchangeably herein, and according to some embodiments, may relate to a glass or plastic container configured to contain and hold a fluid, such as a medication.

The terms “eye” and “eyeball” are used interchangeably herein, and according to some embodiments, may relate to the whole eye, i.e., the approximately globular capsule of a human optical sensor formed by the sclera and cornea together with their contained structures.

The term “about” as used herein according to some embodiments, may refer to plus or minus 10% unless otherwise specified. For example, plus or minus×mm means between 0.9× and 1.1×mm.

Unless specifically stated otherwise, as apparent from the following discussions, it is appreciated that throughout the specification discussions utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

While the invention has been described with respect to a limited number of embodiments, it will be appreciated that many variations, modifications and other applications of the invention may be made. It is therefore intended that the following appended claims and claims hereafter introduced be interpreted to include all such modifications, additions and sub-combinations as are within their true spirit and scope. Therefore, the claimed invention as recited in the claims that follow is not limited to the embodiments described herein.

1.-37. (canceled)

38. An eye syringe, comprising:
an elongated handle comprising a needle that, upon actuation from a retracted position, extends through an opening at a distal end of the handle so as to penetrate an eye of a subject at a predetermined angle, wherein the distal end of the handle comprises a distal section,
the distal end of the distal section forming a concave tip shaped to match and hold an outer surface of an eyeball of the eye,
the distal end of the distal section having a protruding upper edge configured to be inserted behind an upper eyelid so as to distance the upper eyelid from the needle and hold the upper eyelid in place during an injection;
the distal end of the distal section further comprising a lower edge configured to demarcate a border of a portion of the eye so as to accurately position the needle.

39. The syringe of claim 38, wherein the distal section is rotatable about a longitudinal axis of the handle.

40. The syringe of claim 38, wherein the distal section or the handle further comprises a micro-camera configured to capture the border of the portion of the eye.

41. The syringe of claim 38, wherein the lower edge of the distal end of the distal section is configured to demarcate a limbus of the eye, thereby to position the needle at an optimal location for injection at about 3-5 mm from the limbus.

42. The syringe of claim 38, wherein the lower edge of the distal end of the distal section is configured to demarcate an upper portion of the limbus of the eye.

43. The syringe of claim 38, wherein at least one component of the handle or distal section is transparent and is configured to provide a user with a view of the needle, the eyeball or both.

44. The syringe of claim 38, wherein the concave tip is configured to immobilize the eye during the injection and retraction of the needle and/or to stabilize the syringe on the eyeball.

45. The syringe of claim 44, wherein the concave tip comprises a rubber or silicone ring to facilitate immobilization of the eye and/or to stabilize the syringe.

46. The syringe of claim 38, further comprising a needle actuator for actuating the needle.

47. The syringe of claim 46, wherein the needle actuator is protected by a cap.

48. The syringe of claim 38, wherein the handle houses the needle or a portion thereof.

49. The syringe of claim 38, wherein the handle further comprises a port configured to receive a fluid to be injected into the eye.

50. The syringe of claim 38, wherein the handle further comprises a fluid reservoir.

51. The syringe of claim 50, wherein the fluid reservoir is pre-filled with the fluid.

52. The syringe of claim 50, wherein the fluid reservoir is configured to be filled with the fluid through the port.

53. The syringe of claim 38, wherein the handle is configured to receive a vial pre-filled with a fluid to be injected into the eye.

54. The syringe of claim 38, further comprising a movable flap configured to cover a point of injection in a sclera of the eye through which the needle penetrated.

55. The syringe of claim 54, wherein the movable flap is in a first position before the injection and is configured to move to a second position distal to the distal end of the distal section after the injection and retraction of the needle.

56. The syringe of claim 55, wherein in the first position the movable flap is situated on a side portion of the distal end of the distal section.

57. A method of performing an intravitreal injection of medication in an eye, the method comprising:
positioning an eye syringe against the eye so that an upper edge protruding out of a distal end of a rotatable distal section associate with a handle of the eye syringe is inserted under an upper eyelid of the eye, so that the distal end is configured to match a contour of the eye and thereby immobilize an eyeball of the eye, and so that a lower edge of a distal end of the distal section demarcates a limbus of the eye;
actuating an injection of a needle in a handle of the syringe and situated at a predetermined angle for injection through a sclera at a predefined distance from the limbus; and
after injecting through the sclera and retracting of the needle back into the handle.
